(19) United States
(12) Patent Application Publication
(10) Pub. No.: US 2017/0095467 A1
(43) Pub. Date: Apr. 6, 2017

(54) ENCAPSULATION OF ISOQUINOLINE ALKALOIDS

(71) Applicant: Rhizofeed, LLC, Pikeville, KY (US)

(72) Inventors: Wesley G. Barnett, Huddy, KY (US); Molly M. Frank, Greensburg, KY (US); Benjamin T. Clayton, Pikeville, KY (US)

(21) Appl. No.: 15/205,397

(22) Filed: Jul. 8, 2016

Related U.S. Application Data
(60) Provisional application No. 62/235,925, filed on Oct. 1, 2015.

(51) Int. Cl. A61K 31/4741; A61K 36/66; A23K 40/30; A61K 9/16; A23K 20/163; A23K 20/137; A61K 47/36; A61K 9/50

(52) U.S. Cl. CPC A61K 31/4741; A61K 47/36; A61K 36/66; A61K 9/5005; A61K 9/1682; A23K 20/163; A23K 20/137; A23K 40/30; A61K 2236/30; A61K 2236/17; A61K 2236/15

(57) ABSTRACT

Processes of extracting isoquinoline alkaloids from natural sources and encapsulating the alkaloids in an alginate and inulin matrix are provided. The isoquinoline alkaloid encapsulates are useful as animal feed.

### REFERENCE TO RELATED APPLICATIONS

The present application claims the priority of U.S. provisional application Ser. No. 62/235,925, entitled ENCAPSULATION OF ISOQUINOLINE ALKALOIDS, filed Oct. 1, 2015, and hereby incorporates the same application herein by reference.

### TECHNICAL FIELD

The present disclosure generally relates to the extraction and encapsulation of isoquinoline alkaloids. The encapsulated isoquinoline alkaloids can act as a bioactive agent in animal feed.

### BACKGROUND

Isoquinoline alkaloids are known bioactive agents with biological effects on both humans and animals. Although presently considered harmful by the FDA, isoquinoline alkaloids have potential to act as a desirable bioactive agent in applications such as animal feed. Attempts to develop further uses for isoquinoline alkaloid, however, have been hindered by various issues including difficulties in extracting large quantities of the isoquinoline alkaloids and difficulties in protecting the easily damageable alkaloids from the environment following extraction. It would therefore be desirable to provide an improved mechanism to extract, store, and deliver isoquinoline alkaloids for use as a bioactive agent.

### BRIEF DESCRIPTION OF THE DRAWINGS

FIG. 1 depicts a graph illustrating the encapsulation efficiency of a crosslinking process according to one embodiment.

FIG. 2 depicts a representative chromatogram for sanguinarine according to one embodiment.

FIG. 3 depicts a representative chromatogram for chelerythrine according to one embodiment.

FIG. 4 depicts a representative chromatogram for bloodroot extract according to one embodiment.

FIG. 5 depicts a graph illustrating the photodegradation of isoquinoline alkaloids according to one embodiment.

### DETAILED DESCRIPTION

Isoquinoline alkaloids have demonstrated potential to act as a bioactive agent on both internal and external human health. However, the usefulness of isoquinoline alkaloids is not limited to humans and its applicability to animals is increasingly being established. In certain embodiments, isoquinoline alkaloid can be used as a feed additive supplement to, for example, increase the feed conversion ratio (“FCR”) of poultry. In certain embodiments, isoquinoline alkaloid extracts can be used to inhibit the growth of Escherichia coli in poultry.

According to certain embodiments, isoquinoline alkaloid microcapsules can be used as a feed additive in relatively low quantities such as, for example, quantities that are low enough that make it impossible for poultry to ingest quantities high enough to cause bodily harm. Although considered harmful by the FDA, the isoquinoline alkaloid sanguinarine has an LD₅₀ of 1658 mg/kg in rats and natural isoquinoline alkaloid extracts from natural plant sources have LD₅₀ values in the range of 1440 mg/kg to 1250 mg/kg. Potential use of isoquinoline alkaloid microcapsules as a bioactive feed extract is suggested because the bioactive effects of the alkaloids can be demonstrated at levels too low to cause bodily harm to poultry.

The isoquinoline alkaloid microcapsules as disclosed herein can also be useful in combination with other feed additives such as antioxidants or nutrients. For example, isoquinoline alkaloid microcapsules can be useful in combination with phenolic acids, flavonoids, stilbenes, lignans, ethoxyquin, essential oils, oregano, cinnamon, lemon grass, canola, soybean, calcium, phosphorous, corn, wheat, barley, synthetic antioxidants such as BHT and BHQ, vitamins (including A, D, E, K, and B-vitamins (e.g., B12, biotin, folacin, niacin, pantothenic acid, pyridoxine, riboflavin, and thiamin)), microminerals and macrominerals. Examples of microminerals compounds are compounds including copper, iodine, iron, manganese, selenium, or zinc. Examples of macromineral compounds are compounds including calcium, phosphorus, chlorine, magnesium, potassium, or sodium.

According to certain embodiments, isoquinoline alkaloids can be extracted from a natural source. For example, in certain embodiments, isoquinoline alkaloids can be extracted from Sanguinaria canadensis (commonly known as bloodroot). In such embodiments, isoquinoline alkaloids can be extracted from either wild bloodroot or commercial bloodroot. However, wild bloodroot can be preferred in certain embodiments because it has a higher alkaloid content (e.g., about 4% to about 7%) than commercially cultivated bloodroot (about 2% to about 4%). Bloodroot indigenous to Appalachia can be used in certain embodiments.

Non-limiting examples of isoquinoline alkaloids that can be extracted from bloodroot can include benzophenanthridine alkaloids, sanguinarine and chelerythrine. Sanguinarine and chelerythrine, reproduced in formulas (I) and (II) below, are the two alkaloids most prevalent in bloodroot. The minute fibrous roots of bloodroot contain an insignificant quantity of alkaloids. Both sanguinarine and chelerythrine can have similar bioactive effects on humans and animals.

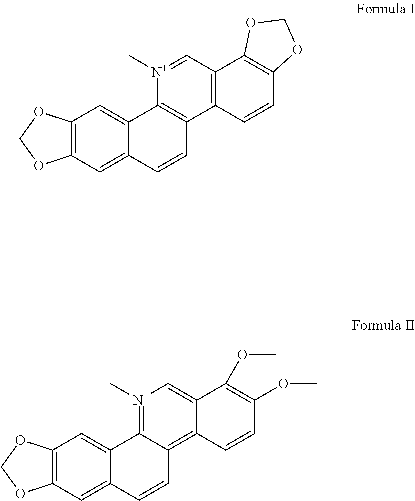 [SMILES: C[n+]1cc2c3c(ccc2c2ccc4cc5c(cc4c21)OCO5)OCO3 ; COc1ccc2c(c[n+](C)c3c4cc5c(cc4ccc23)OCO5)c1OC]

A stable isoquinoline alkaloid microcapsule product can generally be produced through the steps of extracting the isoquinoline alkaloid, and encapsulating the isoquinoline alkaloids in a protective capsule.

According to certain embodiments, an extraction process can include the steps of preparing the isoquinoline alkaloid source and extracting the alkaloids with a solvent. The step of preparing the isoquinoline alkaloid source can generally include the steps of drying the biological source and grinding the source to produce a fine powder. A crude extract of isoquinoline alkaloid can then be extracted from the dried powder with a suitable solvent such as methanol. In certain embodiments extracting alkaloids from bloodroot, about 200 mL of methanol can be used per gram of the powdered bloodroot. Isoquinoline alkaloid sources can generally be dried by placing the source in an oven at elevated temperatures (e.g., about 60° C.) for a sufficient time to dry the sample (e.g., about 24 hours for bloodroot). Dried bloodroot can have approximately 25% of the mass of the undried bloodroot.

Certain steps can be performed to increase the yield of extracted isoquinoline alkaloids. For example, fresh bloodroot can be immediately frozen in a commercial freezer or the like to prevent degradation of the rhizomes. However, even with freezing, damage to the rhizome can still occur with time. As can be appreciated, higher extraction yields can be achieved by immediately extracting the alkaloids. Additional improvements to the extraction yield can also be achieved by grinding the powder and performing any subsequent extraction in the dark due to the photosensitivity of the crude rhizome extract. Exposure to light and UV radiation can degrade the rhizome and can convert the alkaloids to undesirable products.

As can be appreciated, the crude extract of bloodroot and solvent can be further purified in certain embodiments. For example, the crude extract can be filtered through a vacuum filter to remove solid waste. The solid waste can be washed with additional solvent to ensure all of the extracted alkaloids are captured. In certain embodiments, four washings with methanol can be performed. According to certain embodiments, bioactive microcapsules can be formed when the concentration of the isoquinoline alkaloids in the extract are present in concentrations ranging from about 25 parts-per-million (“ppm”) to about 500 ppm in certain embodiments, about 25 ppm to about 250 ppm in certain embodiments, and about 25 ppm to about 100 ppm in certain embodiments. As can be appreciated, excess solvent or additional solvent can be added or removed from to the extract to reach a desired concentration. Excess solvent can be removed through any suitable process such as vacuum evaporation. To avoid damage to the isoquinoline alkaloids, excess solvent can be removed without the use of light or heating of the extract solution to temperatures above about 80° C. in certain embodiments, about 65° C. in certain embodiments, and about 31° C. in certain embodiments.

According to certain embodiments, the isoquinoline alkaloid extract can be mixed with a prebiotic matrix and then crosslinked with alginate and calcium to form encapsulated microbeads of the alkaloids. The encapsulation process can begin with the step of forming a prebiotic matrix of inulin. In certain embodiments, a prebiotic matrix can be prepared by forming a solution of inulin in deionized water. Inulin can be included in any suitable quantity. For example, inulin can be included at a water weight percentage of about 10% to about 32% in certain embodiments, about 20% to about 32% in certain embodiments, and at about 32% in certain embodiments. As can be appreciated, inulin can be fully saturated at a concentration of about 32%. The inulin solution can be heated to elevated temperatures to reach the maximum solubility of inulin in certain embodiments. For example, temperatures of up to about 90° C. can be used to reach a concentration of about 32% water weight percentage inulin.

According to certain embodiments, inulin can be derived from natural sources such as Helianthus tuberosus (commonly referred to as a Jerusalem artichoke). As can be appreciated, such inulin sources are natural carbohydrates suitable for use in biological feed.

In certain embodiments, a prebiotic matrix can alternatively be formed from other carbohydrates including other oligosaccharides and polysaccharides. For example, a prebiotic matrix can alternatively be formed from one or more of mannanoligosaccharide, fructooligosaccharide, and starch.

According to certain embodiments, the isoquinoline alkaloid extract can then be added to the inulin prebiotic matrix, mixed, and allowed to at least partially react with the inulin matrix. In other certain embodiments, the inulin matrix can first be mixed with alginate before the addition of the isoquinoline alkaloid extract.

A microcapsule shell wall can be formed from the crosslinking of alginate and a divalent cation. According to certain embodiments, a solution of about 0.25% to about 5% water weight percentage alginate can be added to the inulin solution.

Microbeads containing the isoquinoline alkaloids can be prepared by crosslinking of the inulin, isoquinoline alkaloid, and alginate solution with a solution of divalent cations. For example, in certain embodiments, the inulin and alginate solution containing the isoquinoline alkaloids can be added dropwise via syringe into a 0.5% water weight calcium sulfate solution that is magnetically stirred at a rate of about 100 rpm or greater. Although calcium sulfate does not readily dissolve in water (Kₛₚ=2.4×10⁻⁵), the calcium ions can still crosslink with the alginate to encapsulate the prebiotic matrix to form microcapsule beads. In certain embodiments, sufficient crosslinking can occur after about 6 minutes. The size of the microcapsule beads can be controlled through selection of needle gauge and pressure. As can be appreciated, needle gauge and pressure can determine the size of the prebiotic matrix droplets.

As can be appreciated, other divalent cation sources can alternatively be used such as, for example, solutions of calcium chloride, magnesium sulfate, calcium sulfate, calcium chloride, magnesium chloride, silver chloride, and certain sulfites. The concentration of the divalent cation source can vary and can be about 0.25% to about 5% in certain embodiments. Cross-linking time can vary depending on the solubility constant of the divalent cation source as well as the concentration of the source.

Following encapsulation, the microcapsule beads can be removed from the solution and dried. Several variations to the drying process are possible. For example, in certain embodiments, the microcapsules can be air dried for about 18 hours to allow the inulin to gel inside the microcapsules. After the inulin has gelled, the microcapsule beads can be placed in a commercial freezer to freeze dry. Freeze drying processes remove additional moisture from the microcapsules and can cause a further reduction in the size of the capsules.

Alternatively, the step of freeze drying can be omitted. In such embodiments, the microcapsules can instead be allowed to air dry for a longer period of time. For example, air drying for a duration of about 72 hours can be sufficient to replicate the effects of freeze drying. Generally, the duration of air drying can be varied depending on the size of the microcapsules as well as the temperature and humidity of the surrounding environment. To avoid damage to the isoquinoline alkaloids, air drying can occur at room temperature (e.g., about 23° C.) in certain embodiments, at about 50° C. or less in certain embodiments, at about 65° C. or less in certain embodiments, and at about 80° C. or less in certain embodiments.

According to certain embodiments, smaller capsules can be preferred in order to increase the bioavailability of the isoquinoline alkaloids. Smaller particles can have a higher impact on metabolism and absorption than coarser or larger particles.

As can be appreciated, the combination of natural storage carbohydrate microbeads followed by the packaging of the resultant microbeads in an enteric-coated capsule provides additional resistance from heat stress during the pelletization process and exposure to environmental conditions while storing. Advantageously, inulin can resist the acidic pH of the stomach and break down in the small intestines.

### EXAMPLES

Encapsulation efficiency was optimized using food grade resveratrol commercially supplied by ChromaDex. As can be appreciated, resveratrol has a known purity and absorbance at 324 nm that allows for very accurate measurement to be made. All measurements were made with a Perkin Elmer Lambda 35 UV/VIS spectrometer using resveratol's λₘₐₓ absorbance band at 324 nm and photodiodes. A slit width of 0.5 nm was used with a path length 121 mm. Table 1 reproduces the standard curve of a resveratrol reference.

TABLE 1

| | Calibration | | STDEV Range | Concentration |
|---|---|---|---|---|
| Standard | Curve | r² | for Absorbance | Range |
| Resveratrol | Y = 0.1059x + | 0.998 | 0.005-0.020 | 2.0-16.0 μg/mL |
| | 0.014 | | | |

To optimize the encapsulation process, the concentration of alginate as well as the storage conditions prior to freeze drying were varied to determine which set of variables would facilitate the highest loading capacity of the alginate beads. As reproduced in Table 2, six different lots were produced from a parent water-based solution that contained 35% water weight inulin and 400 μg/mL resveratrol. Three of the lots included 1% water weight alginate, and three lots included 2% water weight alginate. The beads were formed in accordance with the freeze dry procedure described herein. The beads were air dried for 5 hours before undergoing a freeze-drying process. The beads were placed in the freeze-drying apparatus for a duration of 36 hours using the temperatures depicted in Table 2. Upon removal, each lot of beads were ground into a fine powder and the resveratrol was extracted using methanol.

TABLE 2

| Alginate | Storage | | Dilution | Calculated |
|---|---|---|---|---|
| Concentration | Temperature | Absorbance | Factor | Concentration |
| 1% | −80 Celsius | 1.679 | 80x | 1267 |
| 1% | −20 Celsius | 1.472 | 200x | 2777 |
| 1% | 25 Celsius | 0.905 | 200x | 1706 |
| 2% | −80 Celsius | 0.593 | 80x | 447 |
| 2% | −20 Celsius | 0.715 | 80x | 540 |
| 2% | 25 Celsius | 1.128 | 80x | 851 |

As depicted by Table 2, there is a direct relationship between the loading capacity of the beads and the alginate concentration. As depicted, an alginate concentration of 1% increases the loading capacity by a minimum factor of two. Additionally, the loading capacity can be increased by as much as a factor of 5 when using 1% alginate in comparison to 2% alginate. The decline in the loading capacity with increasing alginate concentration can be caused by the alginate decreasing the free volume within the polymer matrix (a compact structure with smaller pores sizes), and subsequently the amount of analyte that can be entrapped within the pores. This theory is supported by the findings that higher alginate concentration lead to a smaller pore sizes in the resultant microbeads.

The effect of a microencapsulation without alginate was also evaluated. As can be appreciated, inulin exhibits a solubility of 30% in water weight at 90° C. To understand the encapsulation of an inulin only solution, 400 μg/mL of known resveratrol standard was added to a saturated inulin solution without alginate under constant stirring. The inulin was then allowed to fully gel over the course of 24 hours. In the loading study, a 50 mL solution was analyzed. The results are depicted in Table 3.

TABLE 3

| | DI | Gel | | Dilution | Encapsulation |
|---|---|---|---|---|---|
| Inulin | Water | Temp. | Absorbance | Factor | Efficiency |
| 21 g | 70 mL | 25° C. | 0.18, 0.19, | 20x | 85.70% |
| | | | 0.191 | | |

As depicted by Table 3, the efficiency of an encapsulation method using only inulin is higher than that of a comparable encapsulation method including alginate. As can be appreciated, if alginate beads are to be synthesized subsequent to the inulin encapsulation, the inulin encapsulation step can advantageously be allowed to at least partially complete because such encapsulation will reduce subsequent leakage from the alginate beads.

### Crosslinking Time

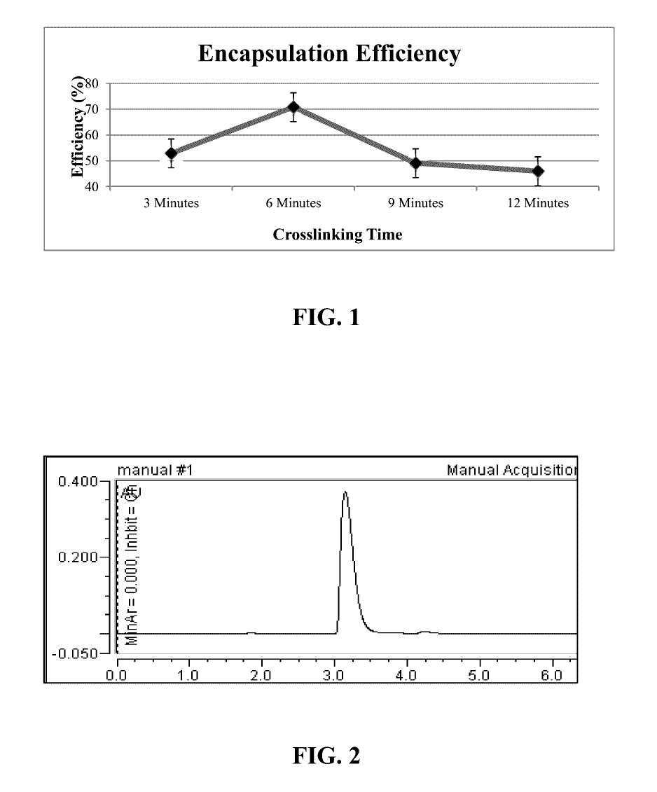

A study was preformed to assess the crosslinking time of the alginate beads in relation to encapsulation efficiency. In the study, a 30% inulin solution was produced at 90° C. with a volume of 70 mL and 400 μg/mL of standard resveratrol. 10 mL aliquots were taken and added dropwise to a 0.5% calcium chloride solution. Crosslinking times of each of the examples were varied using 3 minute intervals. After the respective intervals had elapsed, the beads were removed from the calcium chloride solution, washed with deionized water, dried, and immediately placed in a −20° C. environment for 1 hour. The resulting beads were then freeze-dried for 24 hours and ground into a fine powder. Resveratrol was then extracted from 1 g aliquots of the powders and analyzed with UV-vis spectroscopy to determine the encapsulation efficiency. The spectroscopy results and encapsulation efficiency are depicted in Table 4 and FIG. 1 respectively.

TABLE 4

| | Dry | Sample | | | |
|---|---|---|---|---|---|
| Crosslink | weight | weight | Extraction | Dilution | |
| Time | (g) | (g) | volume | Factor | Absorbance |
| 3 min | 1.95 | 1 | 75 mL | 15.025 | 0.1136 |
| | | | | | 0.1184 |
| 6 min | 2.038 | 1 | 75 mL | | 0.1461 |
| | | | | | 0.1429 |
| 9 min | 1.252 | 1 | 75 mL | | 0.1592 |
| | | | | | 0.1634 |
| 12 min | 1.051 | 1 | 75 mL | | 0.1771 |
| | | | | | 0.18 |

Encapsulation efficiency was derived by comparing the amount of resveratrol that was successfully encapsulated versus the total amount of resveratrol used. Efficiency of the crosslinking step is dependent on the crosslinking time. A certain minimum amount of time is required to form strong microcapsules that do not leak while conversely, too much time produces microcapsules that contain excess alginate and calcium to the exclusion of the alkaloids or resveratrol. As depicted by FIG. 1, the optimal crosslinking time was 6 minutes.

### Optimum Synthesis

Encapsulation of the isoquinoline alkaloids can be reliant on multiple conditions. According to certain embodiments, an advantageous encapsulation process can include the steps of preparing a saturated inulin solution having all inulin fully dissolved with heat at 90° C. The encapsulation process can then continue with the addition of isoquinoline alkaloid to the inulin solution. The inulin solution can then be allowed to partially gel with the isoquinoline alkaloids. A 1% alginate solution can then be added and mixed into the inulin solution. The resultant inulin alginate mixture can then be added dropwise into a 0.5% Ca²⁺ion containing solution (e.g., calcium chloride, calcium sulfate, etc.) and allowed to crosslink for 6 minutes. After 6 minutes, the beads can be promptly removed from solution, rinsed with deionized water, and dried. The beads can then be stored in a −20° C. environment for 1 hour. In such an encapsulation process, freeze-drying can be conducted for 24 hours to produce finished microencapsulated isoquinoline alkaloid beads. The microencapsulated isoquinoline alkaloids can then be stored in a moisture-proof opaque bag or container.

### Quantification

Additional data about the encapsulation methods disclosed herein were verified through high performance liquid chromatography (“HPLC”). Encapsulation efficiency as well as durability of the microcapsules to withstand heat and UV light was also investigated.

### Instrumentation

A Shimadzu LC-20AD Prominence Liquid Chromatograph with DGU-20A3 Shimadzu Prominence Degasser and isocratic pump, SIL-10AF Shimadzu Autosampler, CTO-10AS Shimadzu Column Oven, and CBM-20A Shimadzu Prominence Communications Bus Module was used for the HPLC studies. The identity of peaks was confirmed on a LC-CBM-20A with Dionex Diode Array AD25 Absorbance Detector. Detection was carried out at 330 nm. A XSELECT™HSS T3 (3.5 um, 4.6×150 mm) column was used.

### Reagents and Chemicals

Commercial Pharmco-AAPER Acetonitrile of HPLC grade and Pharmco-AAPER Absolute Anhydrous 200-Proof Ethyl Alcohol of HPLC grade were used for all studies. Standard sanguinarine chloride was acquired from Tocris Bioscience. The commercial sanguinarine chloride was verified by Mass Spectroscopy, ¹NMR, and was determined to have a purity of greater than 97% according to HPLC analysis. Chelerythrine chloride was similarly acquired and tested from TSZ CHEM. The purity of the chelerythrine chloride was greater than 99%.

### HPLC Analysis

A standard solution of sanguinarine chloride was prepared by dissolving 0.01 g of the alkaloid in 10 mL of ethanol and 1 mL of deionized water. 2 mL dilutions of the alkaloid containing 0.1507 mg/mL, 0.2261 mg/mL, and 0.4522 mg/mL of sanguinarine chloride was then prepared by diluting the standard solution in ethyl alcohol with a 0.1M HCl buffer.

A standard solution of chelerythrine chloride was prepared by dissolving 0.01 g of the alkaloid in 20 mL of a solution containing 40% acetonitrile, 59% deionized water, and 1% acetic acid. 2 mL dilutions of the alkaloid containing 0.0313 mg/mL, 0.0625 mg/mL, and 0.1250 mg/mL of chelerythrine was then prepared by diluting the standard solution in distilled water.

2 mL of each sample was then collected. All dilutions were filtered through 0.22 um syringe filter (Restek) and were stored in a dark environment held at −4° C. Mobile phases were prepared using 40% acetonitrile, 59% distilled water, and 1% acetic acid. The flow rate was 1.0000 mL/minute, and a pressure of approximately 2161.06 psi was maintained. UV visible spectroscopy was used to determine the optimum wavelength of detection, and detection was carried out at 330 nm.

Calibration curves based on the three concentrations of sanguinarine chloride and the three concentrations of chelerythrine chloride were obtained by plotting the peak area of the alkaloid versus the concentration. Triplicates were carried out for each dilution. The injection volume of the diluted alkaloid was 10 mL, and each determination was carried out for approximately 5 minutes. Between each dilution determination, a “blank run” (40% acetonitrile, 59% DI water, 1% acetic acid) with an injection volume of 100 mL was carried out for approximately 5 minutes to remove contaminants from the tubing. Relative standard deviation of absorbance was determined to range from 0.0001-0.0011, and relative standard deviation of retention time was determined to range from 0.0030-0.0739.

### Validation Data of Sanguinarine and Chelerythrine in the Bloodroot Rhizome

Table 5 depicts the data from a sample calibration plot and chromatogram. The average retention time, equation of the regression line, correlation coefficient (r²), relative standard deviation range for absorbance, and relative standard deviation range for retention time was collected for each alkaloid. Multiple calibration plots curves were obtained for each alkaloid to ensure accuracy, and all calibration curves exhibited good linear regression.

TABLE 5

| | Average Retention | Calibration | | RSD Range for | RSD Range for |
|---|---|---|---|---|---|
| Alkaloid | Time (min) | Curve | r² | Absorbance | Retention Time |
| Sanguinarine | 3.0437 | Y = 0.7883x + | 0.9997 | 0.0001-0.0011 | 0.0030-0.0035 |
| | | 0.0023 | | | |
| Chelerythrine | 4.1486 | Y = 0.4507x − | 0.9991 | 0.0001-0.0002 | 0.0050-0.0739 |
| | | 0.0057 | | | |

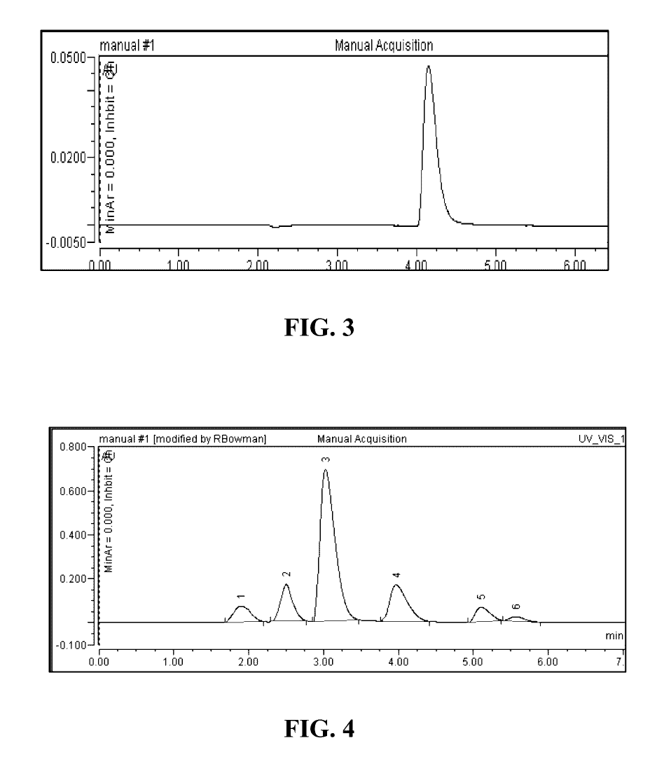

Representative chromatograms for the pure samples of sanguinarine chloride and chelerythrine chloride can be seen in FIGS. 2 and 3 respectively. A representative chromatogram of alkaloids in the extract from the root of Sanguinaria canadensis (Bloodroot) is depicted in FIG. 4. The contents of alkaloids in the extract were calculated using the calibration curves and expressed as a mass percentage of the dried root. Sanguinarine and chelerythrine were the only alkaloids quantified due to their abundancy in bloodroot. Of the total alkaloid percentage, sanguinarine comprises about 50% of the alkaloid content in bloodroot and chelerythrine comprises about 25% of the alkaloid content in bloodroot. Other alkaloids were not quantified. Table 6 depicts the percentage of each alkaloid collected from two sample extractions: one containing bloodroot rhizome without fibrous roots and one containing bloodroot rhizome with fibrous roots.

TABLE 6

| | Percentage of Alkaloid | Percentage of Alkaloid |
|---|---|---|
| | in Extraction Without | in Extraction With |
| Alkaloid | Fibrous Roots | Fibrous Roots |
| Sanguinarine | 5.28% | 5.15% |
| Chelerythrine | 2.92% | 2.99% |
| Total: | 8.19% | 8.14% |

### Optimization of Extraction

### Conditions

To determine the optimal conditions needed to encapsulate a high yield of the alkaloids, various studies were done that varied the light, heat, and extraction time of various encapsulation processes.

### Photosensitivity

Preliminary studies determined that isoquinoline alkaloids are weakly degradable under UV light. In a purified state, it has been established that Sanguinarine undergoes photochemical conversion to oxysanguinarine. To demonstrate that sanguinarine and chelerythrine are photosensitive in crude extracts of the rhizome, HPLC was conducted to determine the effects of short-wave UV light on the extracts.

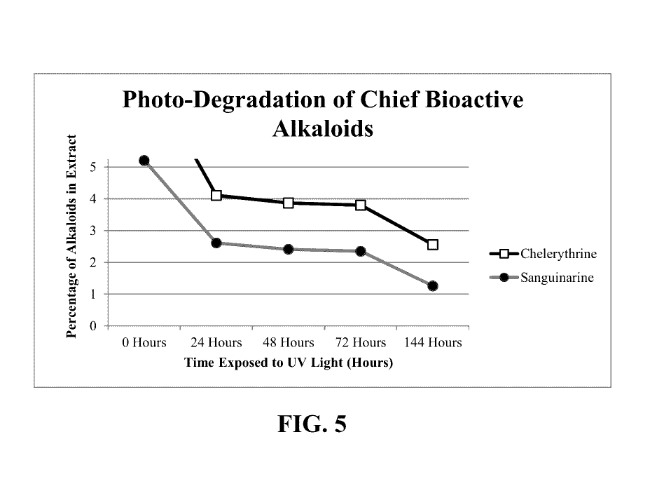

As depicted in Table 7 and FIG. 5, exposure to light or UV radiation over any period of time has a detrimental effect on the encapsulation yield. According to certain embodiments, exposure to light and UV radiation can be minimized by storing the alkaloids in dark conditions until the encapsulation process is complete or the finished product is used.

TABLE 7

| | Percentage of | Percentage of |
|---|---|---|
| | Sanguinarine | Chelerythrine |
| Condition/Duration | in Extract | in Extract |
| Standard Extraction | 5.21% | 2.95% |
| (No Heat or Light Exposure) | | |
| Crude Extract Exposed | 2.61% | 1.50% |
| to UV Light (24 Hours) | | |
| Crude Extract Exposed | 2.41% | 1.46% |
| to UV Light (48 Hours) | | |
| Crude Extract Exposed | 2.35% | 1.45% |
| to UV Light (72 Hours) | | |
| Crude Extract Exposed | 1.26% | 1.30% |
| to UV Light (144 Hours) | | |

### Heat

Two preliminary studies involving heat were conducted. In one study, the raw bloodroot powder was placed on a heating element for 24 hours while the alkaloid was extracted. The alkaloid content in the encapsulates was approximately 27% lower after this heating. In a second study, a crude extract of alkaloids was placed on a heating element at 67° C. to test for degradation. After 24 hours, the total alkaloid concentration dropped from 0.30725 mg/mL to 0.22900 mg/mL indicating that the alkaloids had degraded by approximately 25%. As can be appreciated, heat is a primary cause of alkaloid degradation and any excess heat during an extraction process can lower the final yield as depicted in Table 8.

TABLE 8

| | Percentage of | Percentage of |
|---|---|---|
| | Sanguinarine | Chelerythrine |
| Conditions | in Extract | in Extract |
| Standard Extraction (No Heat or | 5.21% | 2.95% |
| Light Exposure) | | |
| Crude Extract Exposed to Heat | 4.28% | 1.81% |
| (24 Hours) | | |
| Extracted with Heat (24 Hours) | 3.41% | 2.43% |

### Extraction Time

Preliminary studies to acquire optimal extraction time were conducted for 6, 12, 18, and 24 hours. The percentage of alkaloids extracted has shown to be dependent on extraction time. The extraction time with the highest yield was 24 hours in duration.

TABLE 9

| Standard | Percentage of | Percentage of |
|---|---|---|
| Extraction | Sanguinarine | Chelerythrine |
| Duration | in Extract | in Extract |
| 24 Hours | 5.21% | 2.95% |
| 18 Hours | 4.34% | 1.95% |
| 12 Hours | 4.27% | 1.91% |
| 6 Hours | 4.00% | 1.87% |

### Encapsulate Degradation

The optimized encapsulation method disclosed herein was employed to produce alkaloid loaded inulin beads. These beads were subjected to both heat and light to determine the protective properties conferred by the microencapsulation process.

### Heat

To determine the protection the microencapsulation process provides against heat, 1 g of inulin beads was placed in a Fischer Scientific model 605G Isotemp Oven for 24 hours at 96° C. A comparative example of sanguinarine standard that was not encapsulated was also placed in the oven.

A second study was also performed to understand the effect pelletization could have on the microcapsules. In the second study, 1 g of inulin beads was placed in a loosely covered beaker and subjected to 121° C. heat and 16 psi conditions for 20 minutes. These values are much higher in magnitude and duration than will be endured in the pelletization process for animal feed. As depicted in Table 10, the microcapsules decreased degradation by a factor of 1.62.

TABLE 10

| | | Pre-exposure | Post-Exposure | |
|---|---|---|---|---|
| | | Alkaloid | Alkaloid | |
| | | concentration | concentration | Percent |
| Type | Conditions | (mg/mL) | (mg/mL) | Degradation |
| Standard | 96° C., | 0.307 | 0.229 | 25.40% |
| | 24 h | | | |
| Encapsulate | 121° C., | 0.0713 (Sang. | 0.0434 (Sang. | 39.10% |
| | 16 psi, | Only) | Only) | |
| Standard | 24 h | .2002 (Sang. | .07306 (Sang | 63.30% |
| | | Only) | Only) | |

### Light

To determine the light protection provided by the microencapsulation process, 1 g of inulin beads was exposed to a UV source (115V Arthur H. Thomas Co. Lamp). As depicted in Table 11, the microencapsulation process provided substantially increased protection from light degradation.

TABLE 11

| | | Pre-exposure | Post-Exposure | |
|---|---|---|---|---|
| | | Alkaloid conc. | Alkaloid conc. | Percent |
| Type | Conditions | mg/mL | mg/mL | Degradation |
| Standard | UV Light | 0.307 | 0.154 | 49.80% |
| Encapsulate | | 0.119 | 0.083 | 30.70% |

### Results

### Antimicrobial Effects

The antimicrobial activity of the chief isoquinoline alkaloids, sanguinarine and chelerythrine, extracted from the Bloodroot rhizome was evaluated with Escherichia coli (“E. coli”). Diseases caused by the bacteria species E. coli are generally referred to as colibacillosis. Although E. coli can be found in the digestive tract of poultry, most strains are considered to be non-pathogenic. However, a small number of virulent serotypes of E. coli can cause poultry disease. Although widespread on non-organic farms, antibiotic resistant E. coli have been observed on organic poultry farms. In order to reduce the development of antibiotic resistant bacteria, the extracted isoquinoline alkaloids are aimed to act as a phytobiotic alternative to antibiotics in poultry. Although their main method of action is to serve as an alternative attachment site for the colonization of bacteria, its bacteriostatic properties may have relevant applications.

### Methods

30 grams of Tryptic Soy Broth acquired from Carolina Biological Supply Co. was dissolved in one liter of distilled water to produce culture media. Asepsis of all materials was achieved by the use of a LMS Passport autoclave and the conducting of sterile technique throughout the study. Materials were subjected to high-pressure saturated steam at 121° C. for approximately 20 minutes and were stored in a sterilized autoclave bag until desired for use. The bloodroot extract to be evaluated for antimicrobial activity was not subjected to autoclaving due to the thermo-sensitivity of the bioactive alkaloids. Methanol was removed from the crude bloodroot extract via rota-vaporization, and the remaining alkaloids were dissolved in distilled water to a concentration of 2.0339 μg/μL. A proportional amount of Tryptic Soy Broth was dissolved into the solution of alkaloids.

### Screening for Antimicrobial Potential of the Chief Bioactive Isoquinoline Alkaloids

500 μL of Escherichia coli acquired from Carolina Biological Supply Co. was grown in 50 mL of Tryptic Soy Broth at 37° C. in an incubator rotating at 325 rpm. Using a spectrophotometer (600 nm) to evaluate initial turbidity, the turbidity of the bacterial suspensions was adjusted to 0.5 McFarland Standard. 3.3 μL of the bacterial suspension was then placed in each tube containing nutrient broth supplemented with varying concentrations of the bloodroot extract ranging from 5 to 2000 μg of alkaloids. The final concentration of bacteria in each sample was 5×10⁵ cfu/mL. To test the efficiency of the methodology, 3.3 μL of bacteria were also placed in a tube containing 1 mL of pure nutrient broth as a control for comparison.

After 24 hours of rotating incubation (325 rpm) at 37° C., the control containing only nutrient broth and bacteria, and the examples containing nutrient broth, bloodroot extract, and E. coli were centrifuged for 3 minutes at 16,000 rpm. A Minimum Inhibitory Concentration (“MIC”) of the extract was determined by the presence/absence of an E. coli concentrated sediment and is depicted in Table 12. Exponential growth of E. coli caused a sediment to appear on control samples and samples containing an insufficient concentration of the bloodroot. In samples meeting the MIC standard, growth of E. coli was inhibited and no sediment was formed.

TABLE 12

| Concentration (μg/mL) | E. coli |
|---|---|
| 1000 | + |
| 1100 | + |
| 1200 | + |
| 1300 | + |
| 1400 | + |
| 1500 | + |
| 1600 | + |
| 1700 | + |
| 1800 | − |
| 1900 | − |
| 2000 | − |

The MIC of a bloodroot extract with Escherichia coli was determined to be approximately 1800 μg/mL of sanguinarine.

The dimensions and values disclosed herein are not to be understood as being strictly limited to the exact numerical values recited. Instead, unless otherwise specified, each such dimension is intended to mean both the recited value and a functionally equivalent range surrounding that value.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross-referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in the document shall govern.

The foregoing description of embodiments and examples has been presented for purposes of description. It is not intended to be exhaustive or limiting to the forms described. Numerous modifications are possible in light of the above teachings. Some of those modifications have been discussed and others will be understood by those skilled in the art. The embodiments were chosen and described for illustration of various embodiments. The scope is, of course, not limited to the examples or embodiments set forth herein, but can be employed in any number of applications and equivalent articles by those of ordinary skill in the art. Rather it is hereby intended the scope be defined by the claims appended hereto.

What is claimed is:

1. A method of forming a feed supplement, the method comprising:
mixing one or more isoquinoline alkaloid compounds with a prebiotic matrix and alginate to form an intermediate mixture; and
crosslinking the intermediate mixture with a solution of divalent cations to form microbeads of a feed supplement.

2. The method of claim 1, further comprising the step of extracting the one or more isoquinoline alkaloid compounds from a natural source prior to mixing to form the intermediate mixture.

3. The method of claim 2, wherein the step of extracting the one or more isoquinoline alkaloid compounds comprises drying the natural source to form a dried natural source, grinding the dried natural source to produce a dried powder, and exacting the one or more isoquinoline alkaloid compounds from the dried powder with a solvent.

4. The method of claim 2, wherein the step of extracting the one or more isoquinoline alkaloid compounds occurs in a substantially dark environment.

5. The method of claim 2, wherein the step of drying the natural source includes heating the natural resource.

6. The method of claim 2, wherein the concentration of the one or more isoquinoline alkaloid compounds in the solvent is about 25 parts-per-million (“ppm”) to about 500 ppm.

7. The method of claim 2, wherein the natural source is bloodroot.

8. The method of claim 1, wherein the prebiotic matrix and the alginate are mixed together prior to the step of mixing the one or more isoquinoline alkaloid compounds with the prebiotic matrix and the alginate.

9. The method of claim 1, wherein the one or more isoquinoline alkaloid compounds are mixed with the prebiotic matrix prior to addition of alginate, and wherein the one or more isoquinoline alkaloid compounds and the prebiotic matrix partially gel prior to the addition of alginate.

10. The method of claim 1, wherein the one or more isoquinoline alkaloid compounds comprise one or more of sanguinarine and chelerythrine.

11. The method of claim 1, wherein the prebiotic matrix comprises one or more oligosaccharides and polysaccharides.

12. The method of claim 11, wherein the prebiotic matrix comprises a polysaccharide and wherein the polysaccharide comprises inulin.

13. The method of claim 11, wherein the one or more oligosaccharides and polysaccharides comprise mannanoligosaccharide, fructooligosaccharide, and starch.

14. The method of claim 1, further comprising the step of drying the microbeads by one or more of air drying and freeze drying.

15. The method of claim 1, wherein the feed supplement increases the feed conversion rate of poultry.

16. A crosslinked microcapsule comprising:
one or more isoquinoline alkaloid compounds;
one or more oligosaccharides and polysaccharides; and
alginate.

17. The microcapsule of claim 16, wherein the one or more isoquinoline alkaloids comprise sanguinarine and chelerythrine.

18. The microcapsule of claim 16 reduces the degradation of the one or more isoquinoline alkaloid compounds to heat, light, and pressure.

19. The microcapsule of claim 16, further comprising one or more antioxidants and nutrients.

20. The microcapsule of claim 16, further comprising an enteric coating surrounding the crosslinked microcapsule.
